(19) United States
(12) Patent Application Publication
(10) Pub. No.: US 2025/0114365 A1
(43) Pub. Date: Apr. 10, 2025

(54) TOFACITINIB CITRATE SUSTAINED-RELEASE TABLET AND PREPARATION METHOD THEREOF

(71) Applicant: Oryza Pharmaceuticals Shenzhen Limited, Shenzhen (CN)

(72) Inventors: Dacheng TIAN, Shenzhen (CN); Hua Liu, Shenzhen (CN)

(73) Assignee: Oryza Pharmaceuticals Shenzhen Limited, Shenzhen (CN)

(21) Appl. No.: 18/904,685

(22) Filed: Oct. 2, 2024

(30) Foreign Application Priority Data
Oct. 9, 2023 (CN) 202311295239.1

(51) Int. Cl. A61K 31/519; A61K 9/20; A61K 9/28

(52) U.S. Cl. CPC A61K 31/519; A61K 9/2009; A61K 9/2013; A61K 9/2018; A61K 9/2027; A61K 9/2031; A61K 9/2095; A61K 9/2866

(57) ABSTRACT

The disclosure relates to a tofacitinib citrate sustained-release tablet and a preparation method thereof. The tofacitinib citrate sustained-release tablet includes the components in parts by weight: 35.54 parts of tofacitinib citrate, 90-130 parts of diluent, 20-40 parts of binder, 20-40 parts of matrix material, 0.5-1.5 parts of glidant, 1-3 parts of lubricant, and 25.5-53 parts of sustained-release coating material. The sustained-release coating is a mixture of hypromellose, plasticizer and mineral hydrophobic material. The plasticizer is polyethylene glycol. The mineral hydrophobic material is any one of kaolin, talc powder, and heavy density calcium carbonate. Through the combination of a matrix core tablet and the sustained-release coating, a dissolution curve close to that of the existing Xeljanz XR can be achieved, with the sustained-released effect when dissolved in the first 1 h, quickly dissolved within 1-4 h, thereby reducing the production cost, improving the production speed and being environmentally friendly.

### CROSS-REFERENCE TO RELATED APPLICATION

This application claims the benefit of priority to Chinese Patent Application No. 202311295239.1, filed Oct. 9, 2023, the entire disclosure of which is incorporated herein by reference.

### TECHNICAL FIELD

The present disclosure relates to the technical field of pharmacies, and in particular to a tofacitinib citrate sustained-release tablet and a preparation method thereof.

### BACKGROUND

Tofacitinib citrate is a prescription drug for a class of oral Janus kinase (JAK) inhibitors originally developed by Pfizer, can selectively inhibit JAK kinase, block the JAK/STAT pathway, and then inhibit cell signaling transduction as well as related gene expression and activation. A tofacitinib citrate sustained-release tablet is clinically used to treat rheumatoid arthritis, psoriatic arthritis, ankylosing spondylitis, and ulcerative colitis.

The tofacitinib citrate currently on the market includes a fast-release formulation Xeljanz for twice a day, and a sustained-release formulation Xeljanz XR for once a day. Xeljanz XR can reduce the frequency of administration to once a day, decrease fluctuations in blood concentration, and has the same efficacy as Xeljanz for twice a day, which can significantly improve patient convenience and compliance.

Pfizer's sustained-release formulation Xeljanz XR is prepared using an osmotic pump process, an osmotic pressure accelerator such as sorbitol is added in the tablet core except for active ingredients such as tofacitinib citrate and common excipients, a semipermeable membrane coating is coated outside the tablet core, and after coating, one end of the tablet is punched through laser. After the oral Xeljanz XR enters the body, it permeates the semipermeable membrane with the body fluid and enters the tablet core, and then the osmotic pressure of the tablet core rises, such that the active ingredients are extruded through laser holes to release drugs.

The Xeljanz XR preparation process in the existing technology has a complicated process, a relatively high production cost, is not conducive to environmental protection, and has a relatively low productivity. The sorbitol needs to be added in the prescription, this excipient is easy to absorb moisture, so the production workshop needs to be controlled to a low-humidity environment. The semipermeable membrane coating is made of cellulose acetate, which needs to be dissolved in acetone and then coated. The acetone has certain toxicity and is difficult to remove after being coated. The cost of laser punching equipment is relatively high and the drilling speed is relatively slow, which seriously limits the production speed. Moreover, the osmotic pump formulation has inherent defects. As the drug in the semipermeable membrane is released, the osmotic pressure decreases, and the drug cannot be fully released in a later period. Therefore, there is an urgent need for a method for preparing a tofacitinib citrate sustained-release tablet that has a simple preparation process, low production cost, does not require the use of laser punching equipment, has a sustained-release function, and may fully release the drug.

### SUMMARY

In view of the defects in the existing technology, a tofacitinib citrate sustained-release tablet and a preparation method thereof is proposed herein.

The present disclosure provides a tofacitinib citrate sustained-release tablet, including the following components in parts by weight:

| | | |
|---|---|---|
| tofacitinib citrate | 35.54 | parts; |
| diluent | 90-130 | parts; |
| binder | 20-40 | parts; |
| matrix material | 20-40 | parts; |
| glidant | 0.5-1.5 | parts; |
| lubricant | 1-3 | parts; |
| sustained-release coating material | 25.5-53 | parts; |

wherein the sustained-release coating is a mixture of hypromellose, a plasticizer, and a mineral hydrophobic material, wherein the plasticizer is polyethylene glycol, and the mineral hydrophobic material is any one or more of kaolin, talc powder, and heavy density calcium carbonate. A hypromellose polymer material serves as a binder or a film-forming material. The mineral hydrophobic material serves as a coating anti-sticking agent and improves the hydrophobicity of the coating film. The viscosity of the hypromellose is 20-300 cp, such as 35-80 cp (the test method refers to the methods of four parts in ChP (Chinese Pharmacopoeia) 2020 version). The particle size of the mineral hydrophobic material is 500-2,000 meshes (the test method refers to 0982 particle size and particle size distribution determination method in ChP (Chinese Pharmacopoeia) 2020 version).

Further, the diluent is any one or more of microcrystalline cellulose, lactose, a pregelatinized starch, mannitol, and calcium hydrogen phosphate dihydrate.

Further, the binder is any one or more of povidone K29/32 and hydroxypropyl cellulose LF.

Further, the matrix material is any one or more of hypromellose K100LV, hypromellose K4M, hydroxypropyl cellulose GXF, hydroxypropyl cellulose EXF, ethyl cellulose, carbomer 971P NF, and polyoxyethylene.

Further, the glidant is colloidal silicon dioxide.

Further, the lubricant is any one or more of stearic acid and magnesium stearate.

Further, the weight part of the sustained-release coating is 32-50 parts.

Further, in the sustained-release coating, the weight part of the hypromellose is 20-30 parts, the weight part of the polyethylene glycol is 0.5-3 parts, and the weight part of the mineral hydrophobic material is 5-20 parts.

Further, the weight-average molecular weight of the plasticizer is 2,000-8,000.

The present invention further provides a method for preparing the tofacitinib citrate sustained-release tablet, including the following steps:

S1: sieving tofacitinib citrate, diluent, and matrix material one time, mixing them in a granulation pan, and then adding a binder solution for granulation.

S2: after granulation, using a fluidized bed for drying, de-lump the dried granules with a cone mill sieve, then add a glidant and a lubricant to mix to compress into a core tablet; and

S3: preparing a coating solution with a sustained-release coating material, coating the core tablet, and after coating, curing the coated tablet in an oven.

Further, in step S2, the dry air inlet temperature of the fluidized bed is 50-70° C.; and the diameter of the cone mill sieve is 1.0-2.0 mm.

Further, in step S3, the solid concentration of the coating solution is 5%-10%; the tablet temperature during coating is 32-40° C., priority 35-38° C. the solid concentration of the coating solution is: (the weight of solids in the coating solution/the weight of the coating solution)×100%; the curing temperature after coating is 50-70° C., priority 55-65° C.; and the aging time after coating is 12-36 h, priority 16-24 h.

In conclusion, compared with the existing technology, the present invention achieves the following technical effects:

(1) In the present invention, a dissolution release effect similar to Xeljanz XR is achieved in a manner of coating a sustained-release coating outside a matrix core tablet, the active ingredients such as the tofacitinib citrate, diluent, binder and matrix material are granulated, the glidant and lubricant are added to be made into the core tablet, and then the core tablet is coated with the sustained-release polymer coating material. The sustained-release polymer coating material is made of a mixture of the hypromellose, plasticizer, and mineral hydrophobic material. The hypromellose polymer material to act as the binder or film-forming material, and the mineral hydrophobic material to act as the anti-sticking agent and improves the hydrophobicity of the coating film. In the existing technology, as the osmotic pump formulation, Xeljanz XR enters the table core through the semipermeable membrane during dissolution, forming a relatively high osmotic pressure in the tablet core, and the osmotic pressure extrudes the tablet core through the small holes on the side of the formulation, thereby releasing the drug. Therefore, Xeljanz XR releases less drug in the first 1 h. The sustained-release coating in the present disclosure combines the polymer material with the mineral hydrophobic material, in a coating process, the coating material continuously deposits, solidifies, and forms a film on one side, forming a layer of dense and uniform coating. After coating, the tablet is placed in an oven at a certain temperature for curing, which can better promote the integrity, density, and uniformity of the coating film. During dissolution, the sustained-release coating can prevent the penetration of the medium into the tablet core and the dissolution of the drug in the tablet core. As the sustained-release coating continues to corrode and fall off, the drug is released after the tablet core is finally exposed, thus achieving the purpose of sustained release for about 1 h.

(2) The method for preparing the tofacitinib citrate sustained-release tablet in the present invention does not require the use of the laser punching equipment, can reduce the production cost and increase the production speed.

(3) The method for preparing the tofacitinib citrate sustained-release tablet in the present invention does not require the use of sorbitol and acetone, doesn't have low and narrow humidity range requirements in the production workshop, and is environmentally friendly.

### BRIEF DESCRIPTION OF DRAWINGS

In order to more clearly illustrate the technical solutions of the examples of the present disclosure, the drawings required to be used in the examples will be briefly introduced below. It should be understood that the following drawings only show some examples of the present invention and therefore should not be regarded as a limitation to the scope. For those of ordinary skill in the formulation research part, other relevant drawings can be obtained based on these drawings without exerting creative efforts.

FIG. 1 shows a core tablet and a sustained-release coating tablet prepared in Example 1 of the present disclosure and RLD (Reference listed drug) product dissolution curve; the horizontal axis represents time (h), and the vertical axis represents cumulative release rate (%).

### DETAILED DESCRIPTION

In order to enable those formulation researchers to better understand the solutions of the present invention, the technical solutions in the examples of the present invention will be clearly and completely described below in conjunction with the drawings in the examples of the present invention. Obviously, the described examples are only some examples of the present invention, rather than all examples. Based on the embodiments of the present invention, all other examples obtained by those of formulation researcher without creative efforts should fall within the scope of protection of the present invention.

### EXAMPLES

The present invention will be further described below in conjunction with specific examples and comparative examples. The following specific examples are all preferred embodiments of the present invention. However, the embodiments of the present invention are not limited by the following examples, and is not specifically limited by the types of various component raw materials used in the following specific examples.

### I. The Raw Material Sources of the Examples and Comparative Examples are as Follows:

Tofacitinib citrate: lot number TFBB-707-221107, Nantong Chanyoo Pharmatech Co., Ltd.;

Diluent #1: microcrystalline cellulose, model number 101, manufacturer JRS;

Diluent #2: mannitol, model number M100, manufacturer Merck;

Binder #1: hydroxypropyl cellulose, model number LF, manufacturer Ashland;

Binder #2: povidone, model number K29/32, manufacturer Ashland;

Matrix material #1: ethyl cellulose, Viscosity: 10 cp, manufacturer DOW;

Matrix material #2: carbomer, model number 971P NF, manufacturer Lubrizol;

Glidant: colloidal silicon dioxide, the same material was used in other example experiments;

Lubricant: stearic acid, the same material was used in other example experiments;

Hypromellose #1: model number E50LV, viscosity 50 cp, manufacturer DOW;

Hypromellose #2: model number E30, viscosity 30 cp, manufacturer Huzhou Zhanwang;

Hypromellose #3: model number K100LV, viscosity 100 cp, manufacturer DOW;

Hypromellose #4: model number E6LV, viscosity 6 cp, manufacturer DOW;

Plasticizer: polyethylene glycol #1, model number D684L7DBG1, weight-average molecular weight 4,000, manufacturer DOW;

Plasticizer: polyethylene glycol #2, model numberD210K3GJD2, weight-average molecular weight 400, manufacturer DOW;

Mineral hydrophobic material: kaolin #1: model number R116001/NA, particle size 1,250 meshes, manufacturer MLA;

Mineral hydrophobic material: kaolin #2: model number KN-29C102020, particle size 300 meshes, manufacturer MLA;

Mineral hydrophobic material: talc powder: model number Pharma M, particle size 1,250 meshes, manufacturer Imerys;

Mineral hydrophobic material: heavy density calcium carbonate: model number R020136, particle size 1,250 meshes, manufacturer SUDEEP.

The method for preparing the tofacitinib citrate sustained-release tablet in Examples 1-9 and Comparative examples 1-5 of the present invention is as follows:

The tofacitinib citrate, diluent and matrix material were sieved one time by a 30-mesh sieve, and after the above materials were mixed in a granulation pan, binder solution was added for granulation. After granulation, a fluidized bed was used to dry at an air inlet temperature of 60° C. until the loss on drying of the granules was less than 3%. The dried granules de-lumped with a sieve mesh pore diameter of 1.5 mm. Then the glidant and lubricant materials were added to be mixed for 5 min. The final mixing powder was compressed into a core tablet. The sustained-release coating material was added to 70% ethanol aqueous solution to prepare a coating solution. The material temperature during coating was 37° C., the coating time was 3.5-5 h, the solid concentration of the coating solution was 7.55%, and the core tablet was coated. After sustained-release of the coating, curing was conducted in the oven, with a curing temperature of 60° C. and the time of 20 h.

TABLE 1
Technical solutions of Examples 1-9 (in parts by weight)

| | Exam- | Exam- | Exam- | Exam- | Exam- | Exam- | Exam- | Exam- | Exam- |
|---|---|---|---|---|---|---|---|---|---|
| | ple 1 | ple 2 | ple 3 | ple 4 | ple 5 | ple 6 | ple 7 | ple 8 | ple 9 |
| Tofacitinib | 35.54 | 35.54 | 35.54 | 35.54 | 35.54 | 35.54 | 35.54 | 35.54 | 35.54 |
| citrate | | | | | | | | | |
| Microcrystalline | 110 | NA | 130 | 110 | 110 | 110 | 110 | 110 | 110 |
| cellulose | | | | | | | | | |
| Mannitol | NA | 90 | NA | NA | NA | NA | NA | NA | NA |
| Hydroxypropyl | NA | 20 | NA | NA | NA | NA | NA | NA | NA |
| cellulose | | | | | | | | | |
| Polyvidone | 30 | NA | 40 | 30 | 30 | 30 | 30 | 30 | 30 |
| Ethyl cellulose | 30 | 20 | NA | 30 | 30 | 30 | 30 | 30 | 30 |
| Carbomer | NA | NA | 40 | NA | NA | NA | NA | NA | NA |
| Colloidal silicon | 1 | 0.5 | 1.5 | 1 | 1 | 1 | 1 | 1 | 1 |
| dioxide | | | | | | | | | |
| Stearic acid | 2 | 1 | 3 | 2 | 2 | 2 | 2 | 2 | 2 |
| Hypromellose | 25 | 20 | 30 | 25 | 25 | NA | NA | 25 | 28 |
| #1 | | | | | | | | | |
| Hypromellose | NA | NA | NA | NA | NA | 25 | NA | NA | NA |
| #2 | | | | | | | | | |
| Hypromellose | NA | NA | NA | NA | NA | NA | 25 | NA | NA |
| #3 | | | | | | | | | |
| Polyethylene | 2 | 0.5 | 3 | 2 | 2 | 2 | 2 | NA | 2 |
| glycol #1 | | | | | | | | | |
| Polyethylene | NA | NA | NA | NA | NA | NA | NA | 2 | NA |
| glycol #2 | | | | | | | | | |
| Kaolin #1 | 10 | 5 | 20 | NA | NA | 10 | 10 | 10 | 20 |
| Talc powder | NA | NA | NA | 10 | NA | NA | NA | NA | |
| Heavy density | NA | NA | NA | NA | 10 | NA | NA | NA | |
| calcium | | | | | | | | | |
| carbonate | | | | | | | | | |

TABLE 2
Technical solutions of Comparative examples (in parts by weight)

| | Comparative | Comparative | Comparative | Comparative | Comparative |
|---|---|---|---|---|---|
| / | example 1 | example 2 | example 3 | example 4 | example 5 |
| Tofacitinib citrate | 35.54 | 35.54 | 35.54 | 35.54 | 35.54 |
| Microcrystalline | 110 | 110 | 110 | 110 | 110 |
| cellulose | | | | | |
| Polyvidone | 30 | 30 | 30 | 30 | 30 |
| Ethyl cellulose | 30 | 30 | 30 | 30 | 30 |
| Colloidal silicon | 1 | 1 | 1 | 1 | 1 |
| dioxide | | | | | |
| Stearic acid | 2 | 2 | 2 | 2 | 2 |
| Hypromellose #1 | 25 | 12.5 | 45 | | 25 |
| Hypromellose #4 | NA | NA | NA | 25 | NA |
| Polyethylene | NA | 1 | 3.6 | 2 | 2 |
| glycol #1 | | | | | |
| Kaolin #1 | 10 | 5 | 18 | 10 | NA |
| Kaolin #2 | NA | NA | NA | NA | 10 |

### Example 10

The composition of the tofacitinib citrate sustained-release tablet and the dosage of each component in this example were the same as those in Example 1. The drying air inlet temperature of the fluidized bed in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 40° C. until the loss on drying of the granules was less than 3%. The dried granules de-lumped with a sieve mesh pore diameter of 1.5 mm. Then the glidant and lubricant materials were added to be mixed for 5 min. The final mixing powder was compressed into a core tablet. The sustained-release coating material was added to 70% ethanol aqueous solution to prepare a coating solution. The material temperature during coating was 37° C., the solid concentration of the coating solution was 7.55%, and the core tablet was coated. After sustained-release of the coating, curing process was conducted in the oven, with temperature of 60° C. and the time of 20 h.

The results show that when the drying air inlet temperature beyond 50-70° C. is adopted for drying for 3 h, the loss on drying is 4.56%, when drying for 3.5 h, the loss on drying is 4.28%, and dryness at 3% or below is difficultly achieved at an inlet air temperature of 40° C.

### Example 11

The composition of the tofacitinib citrate sustained-release tablet and the dosage of each component in this example were the same as those in Example 1. The sieve mesh pore diameter of the granulation in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules de-lumped with a sieve mesh pore diameter of 3 mm. Then the glidant and lubricant materials were added to be mixed for 5 min. The final mixing powder was compressed into a core tablet. The sustained-release coating material was added to 70% ethanol aqueous solution to prepare a coating solution. The material temperature during coating was 37° C., the solid concentration of the coating solution was 7.55%, and the core tablet was coated. After sustained-release of the coating, curing process was conducted in the oven, with temperature of 60° C. and the time of 20 h.

The results show that when the sieve pore diameter beyond 1.0-2.0 mm is used, larger particle agglomerates still exist after granulation, and the purpose of granulation is not achieved. The bulk density of the particles is measured at 0.413 g/mL, the tap density is 0.624 g/mL, the Carr index is calculated as 33.81%, indicating poor fluidity of the particles.

### Example 12

The composition of the tofacitinib citrate sustained-release tablet and the dosage of each component in this example were the same as those in Example 1. The material temperature during coating in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules de-lumped with a sieve mesh pore diameter of 1.5 mm. Then the glidant and lubricant materials were added to be mixed for 5 min. The final mixing powder was compressed into a core tablet. The sustained-release coating material was added to 70% ethanol aqueous solution to prepare a coating solution. The material temperature during coating was 33° C., the solid concentration of the coating solution was 7.55%, and the core tablet was coated. After sustained-release of the coating, curing process was conducted in the oven, with temperature of 60° C. and the time of 20 h.

The results show that when a non-preferred coating temperature is used, the low temperature causes moisture to penetrate into the tablet core during coating. The coating yield exceeds 100%, which is 110.49%, indicating that moisture penetrates into the tablet core, the tablet drying weight loss is LOD=5.36%, and the moisture is too high.

### Example 13

The composition of the tofacitinib citrate sustained-release tablet and the dosage of each component in this example were the same as those in Example 1. The material temperature during coating in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules de-lumped with a sieve mesh pore diameter of 1.5 mm. Then the glidant and lubricant material were added to be mixed for 5 min to be compressed into a core tablet. The sustained-release coating material was added to 70% ethanol aqueous solution. The material temperature during coating was 30° C., the solid concentration of the coating solution was 7.55%, and the core tablet was coated. After sustained-release of the coating, curing was conducted in the oven, with temperature of 60° C. and the curing time of 20 h.

The results show that if the coating temperature beyond 32-40° C. is adopted, the temperature is too low, resulting in one-sided adhesion, incomplete coating film, and unqualified appearance.

### Example 14

The composition of the tofacitinib citrate sustained-release tablet and the dosage of each component in this example were the same as those in Example 1. The curing temperature after coating in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules were de-lumped with a sieve mesh pore diameter of 1.5 mm. Then the glidant and lubricant were added to be mixed for 5 min to be compressed into a core tablet. The sustained-release coating material was added to 70% ethanol aqueous solution. The material temperature during coating was 37° C., the solid concentration of the coating solution was 7.55%, and the core tablet was coated. After sustained-release of the coating, curing was conducted in the oven, with a temperature of 50° C. and the time of 20 h.

The results show that when a non-preferred curing temperature is used and the cross-sectional structure of the coating piece is observed through a microscope, a small number of bubble-like pores can be observed in the coating film, indicating that the coating film has not completely healed and has not reached a stable state. Pores are not observed in Example 1.

### Example 15

The composition of the tofacitinib citrate sustained-release tablet and the dosage of each component in this example were the same as those in Example 1. The curing temperature after coating in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the drying weight loss of the particles was less than 3%. The dried granules were de-lumped with a sieve mesh pore diameter of 1.5 mm. Then the glidant and lubricant material were added to be mixed for 5 min to be compressed into a core tablet. The sustained-release coating material was added to 70% ethanol aqueous solution. The material temperature during coating was 37° C., the solid concentration of the coating solution was 7.55%, and the core tablet was coated. After sustained-release of the coating, curing was conducted in the oven, with temperature of 40° C. and the time of 20 h.

The results show that a curing temperature beyond 50-70° C. is used, the cross-sectional structure of the coating piece is observed through a microscope, a small number of bubble-like pores can be observed in the coating film, indicating that the coating film has not completely healed and has not reached a stable state. Pores are not observed in Example 1.

### Example 16

The composition of the tofacitinib citrate sustained-release tablet and the dosage of each component in this example were the same as those in Example 1. The curing time after coating in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules were de-lumped with a sieve mesh pore diameter of 1.5 mm. Then the glidant and lubricant were added to be mixed for 5 min, to compress a core tablet. The sustained-release coating material was added to 70% ethanol aqueous solution. The material temperature during coating was 37° C., the solid content of the coating solution was 7.55%, and the core tablet was coated. After sustained-release of the coating, curing was conducted in the oven, with temperature of 60° C. and the time of 12 h.

The results show that when a non-preferred aging time is used and the cross-sectional structure of the coating piece is observed through a microscope, a small number of bubble-like pores can be observed in the coating film, indicating that the coating film has not completely healed and has not reached a stable state. Pores are not observed in Example 1.

### Example 17

The composition of the tofacitinib citrate sustained-release tablet and the dosage of each component in this example were the same as those in Example 1. The curing time after coating in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules were de-lumped with a sieve mesh pore diameter of 1.5 mm. Then the glidant and lubricant were added to be mixed for 5 min, to compress a core tablet. The sustained-release coating material was added to 70% ethanol aqueous solution. The material temperature during coating was 37° C., the solid concentration of the coating solution was 7.55%, and the core tablet was coated. After sustained-release of the coating, curing was conducted in the oven, with temperature of 60° C. and the time of 10 h.

The results show that the curing time beyond 12-36 h is used and the cross-sectional structure of the coating piece is observed through a microscope, a small number of bubble-like pores can be observed in the coating film, indicating that the coating film has not completely healed and has not reached a stable state. Pores are not observed in Example 1.

### Example 18

The composition of the tofacitinib citrate sustained-release tablet and the dosage of each component in this example were the same as those in Example 1. The solid concentration in the coating solution in the preparation method was slightly different for the following:

The tofacitinib citrate, diluent and matrix material were sieved one time by a 30-mesh sieve, and after the above materials were mixed in a granulation pan, a binder solution was added for granulation. After granulation, a fluidized bed was used to dry at an air inlet temperature of 60° C. until the loss on drying of the granules was less than 3%. The dried granules were de-lumped with a sieve mesh pore diameter of 1.5 mm. Then the glidant and lubricant were added to be mixed for 5 min, to compress a core tablet. The sustained-release coating material was added to 70% ethanol aqueous solution. The material temperature during coating was 37° C., the solid concentration of the coating solution was 4%, and the core tablet was coated. After sustained-release of the coating, curing was conducted in the oven, with temperature of 60° C. and the time of 20 h.

The results show that when the solid concentration of the coating solution beyond 5%-10% is used, when the solid content of the coating solution is 4%, the concentration is relatively low. In order to achieve the target weight gain, more coating solution needs to be used, and the coating time is too long, extending from the ideal coating time of 3.5-5 h to about 8 h.

### II. Performance Testing Methods

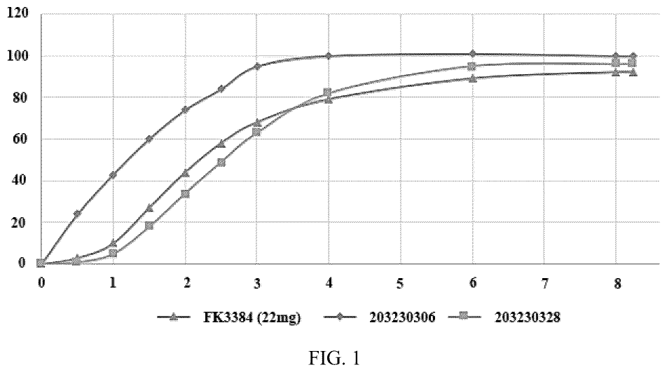

Drug cumulative dissolution release test: the tofacitinib citrate sustained-release tablet prepared in Example 1, the RLD product (lot: FK3384) and the core tablet without coating in Example 1 (i.e., after the compression of the core tablet ends, the subsequent coating step is not required) are tested for the cumulative release at different times. The specific methods are: taking a sample and referring to the release measurement method (Method 2 of Rules of Preparations of Chinese Pharmacopoeia 2015 edition 0931)+sinker device. The dissolution medium is 900 mL of pH 6.8 phosphate buffer, with a rotation speed of 50 rpm, sampling at different times, filtering online, and detecting wavelength at 286 nm. The cumulative dissolution release of the tablet is calculated, and the results are shown in Table 3 (n=10 tablets, representing 10 sample tablets were tested, and the results were averaged). The in vitro drug dissolution release curve is shown in FIG. 1.

TABLE 3
Dissolution data of core tablet, sustained-release coated
tablet and reference formulation

| Time (h) | | | | | | | | | | |
|---|---|---|---|---|---|---|---|---|---|---|
| Batch number | 0 | 0.5 | 1 | 1.5 | 2 | 2.5 | 3 | 4 | 6 | 8 |
| RLD (FK3384) | 0 | 3 | 10 | 27 | 44 | 58 | 68 | 79 | 89 | 92 |
| Core table of Example 1 | 0 | 24 | 43 | 60 | 74 | 84 | 95 | 100 | 101 | 100 |
| (203230306) | | | | | | | | | | |
| Sustained-release coating | 0 | 1 | 5 | 18 | 34 | 49 | 63 | 82 | 95 | 96 |
| tablet of Example 1 | | | | | | | | | | |
| (203230308) | | | | | | | | | | |

The above results show that through the combination of the matrix core table and sustained-release coating, the composition and ratio of Example 1 can achieve a dissolution curve that is closer to that of the RLD product, that sustained-release effect can be achieved if the dissolution is carried out in the first hour, the dissolution is carried out rapidly within 1-4 h, and the sustained-release coating tablet has a dissolution similarity factor f2=63 with the RLD product. The factor f2>50, indicating that the dissolution of the sustained-release coated tablet is similar to that of the RLD product, that is, the quality of Example 1 in the in vitro research is close to that of the RLD product, achieving the research goal of the laboratory stage, and having the conditions for the vivo bioequivalence research for the self-developed samples and the RLD.

The same test is carried out on other examples and comparative examples according to the same method, and the results are shown in Table 4:

TABLE 4
Cumulative dissolution release of examples and
comparative examples (%)

| Time (h) | 0 | 0.5 | 1 | 1.5 | 2 | 2.5 | 3 | 4 | 6 | 8 |
|---|---|---|---|---|---|---|---|---|---|---|
| Example 1 | 0 | 1 | 5 | 18 | 34 | 49 | 63 | 82 | 95 | 96 |
| Example 2 | 0 | 2 | 8 | 23 | 39 | 55 | 69 | 87 | 99 | 101 |
| Example 3 | 0 | 0 | 2 | 13 | 29 | 42 | 57 | 79 | 92 | 95 |
| Example 4 | 0 | 0 | 4 | 17 | 33 | 47 | 61 | 80 | 94 | 97 |
| Example 5 | 0 | 1 | 5 | 19 | 35 | 49 | 64 | 84 | 97 | 100 |
| Example 6 | 0 | 3 | 8 | 21 | 39 | 55 | 66 | 89 | 97 | 100 |
| Example 7 | 0 | 0 | 2 | 14 | 31 | 43 | 58 | 80 | 92 | 96 |
| Example 8 | 0 | 3 | 7 | 24 | 40 | 55 | 70 | 88 | 98 | 102 |
| Example 9 | 0 | 0 | 2 | 14 | 31 | 45 | 60 | 79 | 93 | 96 |
| Comparative | 0 | 4 | 10 | 25 | 42 | 56 | 67 | 85 | 98 | 100 |
| example 1 | | | | | | | | | | |
| Comparative | 0 | 7 | 13 | 28 | 48 | 61 | 72 | 90 | 100 | 101 |
| example 2 | | | | | | | | | | |
| Comparative | 0 | 0 | 1 | 9 | 25 | 40 | 54 | 76 | 92 | 97 |
| example 3 | | | | | | | | | | |
| Comparative | 0 | 11 | 33 | 51 | 63 | 73 | 86 | 99 | 100 | 101 |
| example 4 | | | | | | | | | | |

Compared with Example 1, Example 6 uses hypromellose with a viscosity of 30 cp, the viscosity is relatively low and the release after coating is relatively fast. Compared with Example 1, Example 7 uses hypromellose with a viscosity of 100 cp, the viscosity is too high and the release after coating is too slow. The above examples illustrate that the preferred 35-80 cp hypromellose is more effective. Compared with Example 1, Example 8 used polyethylene glycol beyond the molecular weight range is not as good as that of Example 1, indicating that the effect of using polyethylene glycol with a weight-average molecular weight of 2,000-8,000 is better.

Comparative Examples 1-5 all have the single variable as Example 1. The sustained-release coating material of Comparative example 1 lacks polyethylene glycol, the weight part of the sustained-release coating material mixture of Comparative example 2 is too low, and the weight part of the sustained-release coating material of Comparative example 3 is too high, the weight gain is too high, and the release is too slow. Comparative example 4 uses hypromellose beyond the viscosity range, the viscosity is relatively low and the release speed is too fast, which fails to achieve the purpose of sustained-release coating. Comparative example 5 uses kaolin beyond the particle size range, the particle size is too large, and the prepared coating solution is easy to settle, which will cause the coating gun to block and the not be prepared successfully. The above comparative examples either cannot be successfully prepared or cannot achieve the sustained release performance of the examples, that is, the dissolution is slower than the RLD in the first 3 hours, about 5%-15% slower, and the dissolution curve intersects with the reference in about 3-4 hours, and the dissolution is faster than the RLD about 4 h until the dissolution platform is reached.

Based on the cumulative release test results in Table 4, the tofacitinib citrate sustained-release tablet prepared through Examples 1-9 has a sustained-release effect when being dissolved in the first 1 hour and rapidly dissolved within 1-4 h, which can effectively meet the requirements of high standard demands of customers and market.

The above are only preferred embodiments of the present disclosure and are not intended to limit the present disclosure. Any modifications, equivalent substitutions, improvements, etc. made within the spirit and principles of the present disclosure shall be included in the scope of protection of the present disclosure.

1. A tofacitinib citrate sustained-release tablet, comprising the following components in parts by weight:

| | | |
|---|---|---|
| tofacitinib citrate | 35.54 | parts; |
| diluent | 90-130 | parts; |
| binder | 20-40 | parts; |
| matrix material | 20-40 | parts; |
| glidant | 0.5-1.5 | parts; |
| lubricant | 1-3 | parts; |
| sustained-release coating | 25.5-53 | parts; |

wherein the sustained-release coating is a mixture of hypromellose, a plasticizer and a mineral hydrophobic material;
wherein the plasticizer is polyethylene glycol; and the mineral hydrophobic material is any one or more of kaolin, talc powder, and heavy density calcium carbonate;
the viscosity of the hypromellose is 20-300 cp; and
the particle size of the mineral hydrophobic material is 500-2,000 meshes.

2. The tofacitinib citrate sustained-release tablet according to claim 1, wherein the diluent is any one or more of microcrystalline cellulose, lactose, a pregelatinized starch, mannitol, and calcium hydrogen phosphate dihydrate.

3. The tofacitinib citrate sustained-release tablet according to claim 1, wherein the binder is any one or more of povidone K29/32 and hydroxypropyl cellulose LF.

4. The tofacitinib citrate sustained-release tablet according to claim 1, wherein the matrix material is any one or more of hypromellose K100LV, hypromellose K4M, hydroxypropyl cellulose GXF, hydroxypropyl cellulose EXF, ethyl cellulose, carbomer 971P NF, and polyoxyethylene.

5. The tofacitinib citrate sustained-release tablet according to claim 1, wherein the weight part of the sustained-release coating is 32-50 parts.

6. The tofacitinib citrate sustained-release tablet according to claim 1, wherein in the sustained-release coating, the weight part of the hypromellose is 20-30 parts, the weight part of the polyethylene glycol is 0.5-3 parts, and the weight part of the mineral hydrophobic material is 5-20 parts.

7. The tofacitinib citrate sustained-release tablet according to claim 1, wherein the weight-average molecular weight of the plasticizer is 2,000-8,000.

8. A method for preparing the tofacitinib citrate sustained-release tablet according to claim 1, comprising the following steps:
S1: sieving tofacitinib citrate, a diluent and a matrix material one time, and mixing them in a granulation pan, and then adding a binder solution for granulation;
S2: after granulation, using a fluidized bed for drying, granulating dried granules, then adding a glidant and a lubricant to mix to compress into a core tablet; and
S3: preparing a coating solution with a sustained-release coating material, coating the core tablet, and after coating, curing the coated tablet in an oven.

9. The method according to claim 8, wherein in step S2, the drying air inlet temperature of the fluidized bed is 50-70° C.; and the pore diameter of the granulating sieve mesh is 1.0-2.0 mm.

10. The method according to claim 8, wherein in step S3, the solid concentration of the coating solution is 5%-10%; the material temperature during coating is 32-40° C.; the curing temperature after coating is 50-70° C.; and the curing time after coating is 12-36 h.

11. The method according to claim 8, wherein the diluent is any one or more of microcrystalline cellulose, lactose, a pregelatinized starch, mannitol, and calcium hydrogen phosphate dihydrate.

12. The method according to claim 8, wherein the binder is any one or more of povidone K29/32 and hydroxypropyl cellulose LF.

13. The method according to claim 8, wherein the matrix material is any one or more of hypromellose K100LV, hypromellose K4M, hydroxypropyl cellulose GXF, hydroxypropyl cellulose EXF, ethyl cellulose, carbomer 971P NF, and polyoxyethylene.

14. The method according to claim 8, wherein the weight part of the sustained-release coating is 32-50 parts.

15. The method according to claim 8, wherein in the sustained-release coating, the weight part of the hypromellose is 20-30 parts, the weight part of the polyethylene glycol is 0.5-3 parts, and the weight part of the mineral hydrophobic material is 5-20 parts.

16. The method according to claim 8, wherein the weight-average molecular weight of the plasticizer is 2,000-8,000.
